(19) United States
(12) Patent Application Publication
(10) Pub. No.: US 2011/0100371 A1
(43) Pub. Date: May 5, 2011

(54) ANTI-VIRAL PROPERTIES OF ZOSTERIC ACID AND RELATED MOLECULES

(75) Inventors: Scott F. Michael, Estero, FL (US); Sharon Isern, Estero, FL (US); Joshua Costin, Naples, FL (US)

(73) Assignee: FLORIDA GULF COAST UNIVERSITY, Ft. Myers, FL (US)

(21) Appl. No.: 12/668,847

(22) PCT Filed: Jul. 11, 2008
(86) PCT No.: PCT/US08/69808

§ 371 Date: Nov. 29, 2010

Related U.S. Application Data
(60) Provisional application No. 60/949,694, filed on Jul. 13, 2007.
(60) Provisional application No. 61/058,026, filed on Jun. 2, 2008.

(51) Int. Cl. A62B 7/10; C07C 305/24; C07C 69/732; C07K 14/00; A61K 31/255; A61K 31/216; A61P 31/18; A61P 31/16; A61P 31/14; A01N 41/02; A01N 37/10; A01P 15/00; A01N 25/34; C07K 14/765; B32B 15/04; B32B 9/04; B32B 1/02

(52) U.S. Cl. 128/206.19; 558/32; 560/75; 530/410; 514/517; 514/532; 424/408; 530/363; 428/457; 428/411.1; 428/34.1

(57) ABSTRACT

The invention relates chemical compound entry inhibitors and methods of determining such inhibitors that interact with regions of viruses, such as the dengue virus, as candidates for in vivo anti-viral compounds.

### CROSS REFERENCE

This is a national stage application of PCT/US2008/69808, filed Jul. 11, 2008, to which this application claims priority from and any other benefit of U.S. provisional patent application Ser. Nos. 61/058,026 filed Jun. 2, 2008 and 60/949,694 filed Jul. 13, 2007, which are incorporated herein by reference.

### TECHNICAL FIELD

The invention relates to inhibitors that interact with regions of a virus. More particularly, the invention relates to chemical compounds that act as inhibitors and methods of determining such inhibitors that interact with regions of a virus, as candidates for the development of anti-viral compounds, including in vivo anti-viral compounds.

### BACKGROUND OF THE INVENTION

Biological attachments are ubiquitous and required for the existence of all multicellular life. However, these attachments are also used by parasites and pathogens. The impact of detrimental biological adhesion is broad and involves interactions at multiple levels, including macroscopic encrustation, biofilm formation, and microscopic pathogen-host recognition. In response, organisms have evolved strategies to defend against harmful biological interactions. The temperate marine eelgrass, Zostera marina, produces an anti-adhesive chemical, p-sulfoxy-cinnamic acid, also known as zosteric acid that inhibits colonization of the leaf surfaces by encrusting algae and other organisms. The mechanism of activity is thought to be mediated by binding to, or coating, the encrusting organisms, and subsequent release of the zosteric acid and the organism from the leaf surface. In support of this, solutions of free zosteric acid have been shown to have anti-fouling and anti-adhesion activities against algae, fungal spores, and bacteria. Several groups have also reported anti-adhesive effects against crustacean larvae (barnacles), mollusks, algae, fungal spores, and bacteria by incorporation of zosteric acid into slow-release surface coatings. This wide range of anti-adhesion activity displayed by zosteric acid against such a variety of different organisms suggests a mechanism targeting chemical interactions that are highly conserved in many biological attachment processes.

All organisms with a requirement for biological adhesion in their life cycles must identify and interact with target surfaces and actively distinguish between relevant surfaces and both biological and non-biological non-relevant surfaces. Investigation of virus binding and entry events has led to a generalized multi-step model of “adhesion strengthening”, where initial low affinity, high abundance interactions are followed by high affinity, low abundance specific interactions that lead to target cell entry. Sonic well-characterized examples include the initial interaction of herpes simplex virus with cell surface heparin sulfate and reovirus and influenza virus with sialic acid. DENVs show a similar multi-step process during infection, using interactions with heparin sulfate on mammalian target cells for attachment, although other carbohydrates may also be utilized in certain cells, and infection in insect cells may occur by direct binding to a proteinacious receptor, bypassing interactions with heparin. Direct DENV interactions with secondary protein receptors are diverse between different mammalian cell lines, between DENY types, and between different DENV isolates within the same strain. It is likely that DENV enters target cells by a multi-step binding/recognition mechanism using several different carbohydrate and proteinacious receptors, perhaps in a redundant fashion that may differ between different cell types and DENY strains. Despite the diversity in receptor use, the DENV entry pathway has been identified as a promising target for the development of anti-virals, and there is a need for the development of such anti-virals for the treatment of disease induced by the DENV strains or other viruses.

Together, the four strains of DENY comprise the most common human arboviral infection and the most important public health threat from mosquito-born viral pathogens. Currently, there is no approved vaccine or specific therapy that exists for the prevention or treatment of DENV infection, making DENY an attractive target for the development of inhibitors that demonstrate an anti-viral effect based on the chemistry of zosteric acid and related chemistries.

### SUMMARY OF THE INVENTION

The invention provides chemical compounds that are bindable to regions within different viruses and inhibit the activity of these viruses. The interaction of an inhibitor with such regions, or the modulation of the activity of such regions with an inhibitor, could inhibit viral fusion and hence viral infectivity. In one aspect, the invention provides compounds and methods of screening the compounds against these bindable regions in order to discover therapeutic candidates for a disease caused by a virus. Diseases for which a therapeutic candidate may be screened include dengue fever, dengue hemorrhagic fever, influenza, tick-borne encephalitis, West Nile virus disease, yellow fever, human immunodeficiency virus (HIV) and hepatitis C.

In one embodiment, a method for identifying a therapeutic candidate for a disease caused by a virus includes contacting a bindable region of the virus with a chemical compound, wherein binding of the chemical compound indicates a therapeutic candidate. The chemical compounds may be selected from compounds including zosteric acid and derivatives thereof. Based on the possibility that viruses make interactions similar to other biological adhesives as they target new host cells for infection, the invention provides compounds, including zosteric acid or related chemistries, that possess anti-viral activities. Viruses are structurally much simpler than other cellular microorganisms and, as such, present good systems to examine the interactions of zosteric acid and other chemistries with biologically relevant surface molecules. Binding may be assayed either in vitro or in vivo. In certain embodiments, the virus is the dengue virus, the influenza virus or HIV. Such bindable regions also may be utilized in the structure determination, drug screening, drug design, and other methods described and claimed herein.

In one embodiment, zosteric acid and other chemistries inhibit DENV-2 with fifty percent inhibitory concentration (IC₅₀) in the 2 mM range, another compound inhibits in the 300 μM range, and the most active compound shows an IC₅₀ in the range of 14-47 μM against all of the four strains of DENV. The most active compound functions at an early entry step in the viral life cycle, prior to internalization and fusion, but that it does not prevent virion binding to the target host cell. This represents the first demonstration of an anti-viral effect of zosteric acid and related chemistries.

In another embodiment of the invention, an anti-viral compound includes a chemical compound represented by general structure:

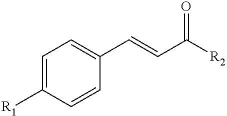

wherein,

R₁ represents —OH or —OSO₂OH;

R₂ represents —OH, optionally substituted alkyl, heteroalkyl, cycloalkyl, heterocycloalkyl, aryl, heteroaryl, aralkyl, or hetero aralkyl.

In yet another embodiment of the invention, a method for inhibiting viral infection includes the steps of contacting a compound within a bindable region of a virus, wherein the compound inhibits fusion between a virion envelope and a cell membrane.

### BRIEF DESCRIPTION OF THE DRAWINGS

FIG. 1A is a graph representing a dose response inhibition for zosteric acid against DENV-2 in focus forming assays;

FIG. 1B is a graph representing a dose response inhibition for CF 238 against DENV-2 in focus forming assays;

FIG. 1C is a graph representing a dose response inhibition for CF 285 against DENV-2 in focus forming assays;

FIG. 1D is a graph representing a dose response inhibition for CF 290 against DENY-2 in focus forming assays;

FIG. 1E is a graph representing a dose response inhibition for CF 296 against DENV-2 in focus forming assays;

FIG. 1F is a graph representing a dose response inhibition for CF 490 against DENV-2 in focus forming assays;

FIG. 2 is a graph representing the dose response inhibition curves for CF 238 against DENV-1,2,3 and 4 in focus forming assays;

FIG. 3A is a graph representing MTT mitochondrial reductase toxicity assay for zosteric acid;

FIG. 3B is a graph representing MTT mitochondrial reductase toxicity assay for CF 238;

FIG. 3C is a graph representing MTT mitochondrial reductase toxicity assay for CF 285;

FIG. 3D is a graph representing MTT mitochondrial reductase toxicity assay for CF 290;

FIG. 3E is a graph representing MTT mitochondrial reductase toxicity assay for CF 296;

FIG. 3F is a graph representing MTT mitochondrial reductase toxicity assay for CF 490;

FIG. 4A is a graph representing a dose response inhibition for CF 238 against DENV-2 in post-entry focus-forming assay;

FIG. 4B is a graph representing a dose response inhibition for CF 238 against DENV-2 in pre-binding focus-forming assay; and

FIG. 5 is a graph representing a qRT-PCR binding assay of DENV-2 to target cells;

### DETAILED DESCRIPTION OF THE INVENTION

Chemical compounds capable of exhibiting inhibitory activity against viruses in cell culture systems are described herein. The chemical compounds were developed through the rational design and synthesis of novel, dimeric chemistries with two symmetrical or non-symmetrical phenolic groups, different length linkers, and modifications to the functional groups found in a compound having the general structure 1:

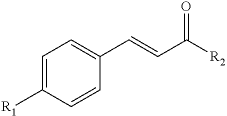

wherein,
R₁ represents —OH or —OSO₂OH;
R₂ represents —OH, optionally substituted alkyl, heteroalkyl, cycloalkyl, heterocycloalkyl, aryl, heteroaryl, aralkyl, or heteroaralkyl.

Chemical compounds having the general structure 1 are represented Table 1. In particular, the inhibitory activity of zosteric acid and selected related chemistries against dengue viruses (DENV) in cell culture systems are described.

TABLE 1

| Name | Chemical Structure | IC₅₀ μM ± sem |
|---|---|---|
| ZA | 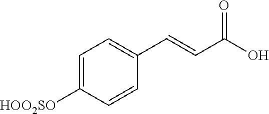 | 2,380 ± 150 |
| CF 238 | 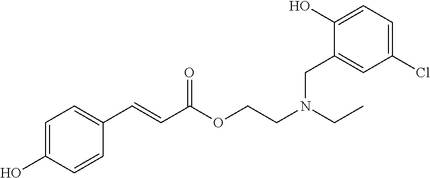 | 24 ± 6 D-1 46 ± 4 D-2 14 ± 2 D-3 47 ± 5 D-4 |
| CF 285 | 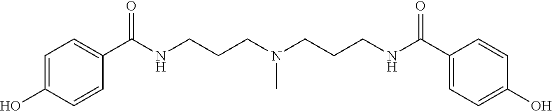 | 2,516 ± 172 |
| CF 290 | 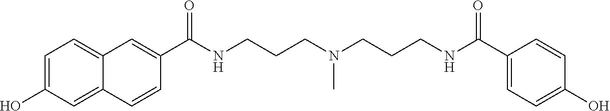 | 294 ± 42 |
| CF 296 | 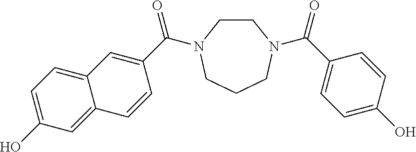 | N/A |
| CF 490 | 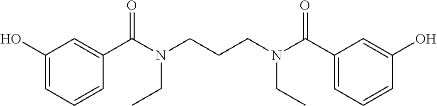 | 2,378 ± 192 |

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., —C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a hydroxyl, a carbonyl (such as a carboxyl, an alkoxycarbonyl, a formyl, or an acyl), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN and the like.

Cycloalkyls can be further substituted with alkyls, alkenyls, alkoxys, alkylthios, aminoalkyls, carbonyl-substituted alkyls, —CN, and the like.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CN, or the like. The term “aryl” also includes polycyclic ring systems having two or more rings in which two or more carbons are common to two adjoining rings (the rings are “fused”) wherein at least one of the rings is aromatic, e.g., the other rings can be cycloalkyls, cycloalkenyls, cycloalkynyls, aryls and/or heterocyclyls.

The term “heteroatom” as used herein means an atom of any element other than carbon or hydrogen. Heteroatoms are nitrogen, oxygen, sulfur and phosphorous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, perimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CN, or the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Alkyl groups are lower alkyls and a substituent designated herein as alkyl is a lower alkyl.

An embodiment of the invention relates to methods of inhibiting dengue infection that includes inhibiting the fusion between the virion envelope and a cell membrane, the process that delivers the viral genome into the cell cytoplasm.

Any chemical compound which inhibits the fusion between the dengue virion envelope and a cell membrane, including those of the dengue virus which infect human as well as nonhuman hosts, may be used according to the invention. In various embodiments of the invention, these chemical compound dengue entry inhibitors may include, but are not limited to zosteric acid and selected related chemistries that are complimentary to several membrane-interactive bindable regions of dengue virus proteins.

The term “bindable region”, when used in reference to a chemical compound, complex and the like, refers to a region of a dengue virus E protein or other class II E protein which is a target or is a likely target for binding an agent that reduces or inhibits viral infectivity. For a chemical compound such as zosteric acid for example, a bindable region generally refers to a region wherein functional groups of the chemical compound would be capable of interacting with at least a portion of the dengue virus E protein. For a chemical compound or complex thereof, bindable regions including binding pockets and sites, interfaces between domains of a chemical compound or complex, surface grooves or contours or surfaces of a chemical compound or complex which are capable of participating in interactions with another molecule, such as a cell membrane.

In other embodiments of the invention, the dengue chemical compound entry inhibitors including related chemistries are linked to a carrier molecule such as a protein. Proteins contemplated as being useful according to this embodiment of the invention, include but are not limited to, human serum albumen. Dengue chemical compound entry inhibitors comprising additional functional groups are also contemplated as useful according to the invention.

The dengue entry inhibitory chemical compounds of the invention may be utilized to inhibit dengue virus virion:cell fusion and may, accordingly, be used in the treatment of dengue virus infection. The chemical compounds of the invention may be administered to patients in any sterile, biocompatible pharmaceutical carrier, including, but not limited to, saline, buffered saline, dextrose, and water. Methods for administering chemical compounds to patients are well known to those of skill in the art; they include, but are not limited to, intradermal, intramuscular, intraperitoneal, intravenous, subcutaneous, oral, and intranasal. In addition, it may be desirable to introduce the pharmaceutical compositions of the invention into the central nervous system by any suitable route, including intravenous injection. Other embodiments contemplate the administration of the dengue entry inhibitory chemical compounds or derivatives thereof, linked to a molecular carrier (e.g. HSA).

A number of techniques can be used to screen, identify, select and design chemical entities capable of associating with a dengue virus E protein or other class II E protein, structurally homologous molecules, and other molecules. Knowledge of the structure for a dengue virus E protein or other class II E protein, determined in accordance with the methods described herein, permits the design and/or identification of molecules and/or other modulators which have a shape complementary to the conformation of a dengue virus E protein or other class II E protein, or more particularly, a druggable region thereof. It is understood that such techniques and methods may use, in addition to the exact structural coordinates and other information for a dengue virus E protein or other class II E protein, and structural equivalents thereof.

In one aspect, the method of drug design generally includes computationally evaluating the potential of a selected chemical compound to associate with a molecule or complex, for example any class II viral E protein. For example, this method may include the steps of employing computational means to perform a fitting operation between the selected chemical compound and a bindable region of the molecule or complex and analyzing the results of the fitting operation to quantify the association between the chemical entity and the bindable region.

In another aspect, potential candidates as dengue chemical compound entry inhibitors of DENV infectivity that target the viral E protein were determined through the use molecular modeling of the dengue chemical compound entry inhibitors in conjunction a Monte Carlo binding algorithm and a Wimley-White interfacial hydrophobicity scale.

The term “Monte Carlo,” as used herein, generally refers to any reasonably random or quasi-random procedure for generating values of allowed variables. Examples of Monte Carlo methods include choosing values: (a) randomly from allowed values; (b) via a quasi-random sequence like LDS (Low Discrepancy Sequence); (c) randomly, but biased with experimental or theoretical a priori information; and (d) from a non-trivial distribution via a Markov sequence.

More particularly, a “Monte Carlo” method is a technique which obtains a probabilistic approximation to the solution of a problem by using statistical sampling techniques. One Monte Carlo method is a Markov process, i.e., a series of random events in which the probability of an occurrence of each event depends only on the immediately preceding outcome. (See Kalos, M. H. and Whitlock, P. A. “Monte Carlo Methods: Volume I: Basics,” John Wiley & Sons, New York, 1986; and Frenkel, D., and Smit, B. “Understanding Molecular Simulation: From Algorithms to Applications,: Academic Press, San Diego, 1996).

The Wimley-White interfacial hydrophobicity scale is a tool for exploring the topology and other features of membrane proteins by means of hydropathy plots based upon thermodynamic principles.

### Materials and Methods

### Synthesis of Chemistries

Five novel chemistries related to ZA were designed by CernoFina, LLC (Portland, Me.) employing a combinatorial approach using phenol or napthol rings in a symmetric or non-symmetric fashion attached together with amine-containing, variable linker regions (See Table 1). ZA and the other chemistries were synthesized and provided by Pittsburgh Plate and Glass Industries (Pittsburgh, Pa.). Chemistries were dissolved in dimethylsulfoxide (DMSO) and diluted into PBS or Dulbecco's modified eagle medium (DMEM) to a final concentration containing 1% or less DMSO.

### Viruses and Cells

DENV-1 strain HI-1, DENV-2 strain NG-2, DENV-3 strain H-78, and DENV-4 strain H-42 were obtained from R. Tesh at the World Health Organization Arbovirus Reference Laboratory at the University of Texas at Galveston. Viruses were propagated in the African green monkey kidney epithelial cell line, LLCMK-2, a gift of K. Olsen at Colorado State University. LLCMK-2 cells were grown in Dulbecco's modified eagle medium (DMEM) with 10% (v/v) fetal bovine serum (FBS), 2 mM Glutamax, 100 U/ml penicillin G, 100 μg/ml streptomycin and 0.25 μg/ml amphotericin B, at 37° C. with 5% (v/v) CO₂.

### Focus Forming Unit (FFU) Reduction Assay

LLCMK-2 target cells were seeded at a density of 1×10⁵ cells in each well of a 6-well plate 24 h prior to infection. Approximately 200 FFU of virus were incubated with or without chemistries in serum-free DMEM for 1 h at rt. Virus/chemistry or virus/control mixtures were allowed to infect confluent target cell monolayers for 1 h at 37° C., with rocking every 15 m, after which time the medium was aspirated and overlaid with fresh DMEM/10% (v/v) FBS containing 0.85% (w/v) Sea-Plaque Agarose (Cambrex Bio Science, Rockland, Me.). Cells with agar overlays were incubated at 4° C. for 20 m to set the agar. Infected cells were then incubated at 37° C. with 5% CO₂ for 3 days (DENV-1, 3 and 4) or 5 days (DENV-2). Infected cultures were fixed with 10% formalin overnight at 4° C., permeabilized with 70% (v/v) ethanol for 20 m, and rinsed with PBS prior to immunostaining. Virus foci were detected using supernatant from mouse anti-DENV hybridoma E60 (obtained from M. Diamond at Washington University) followed by horseradish peroxidase-conjugated goat anti-mouse immunoglobulin (Pierce, Rockford, Ill.) and developed using AEC chromogen substrate (Dako, Carpinteria, Calif.). Results were expressed as the average of at least two independent trials with three replicates in each trial.

### Cytoxicity Assay

The cytotoxicity of the chemistries was measured by monitoring mitochondrial reductase activity using the TACS™ MTT cell proliferation assay (R&D Systems, Inc., Minneapolis, Minn.) according to the manufacturer's instructions. Dilutions of chemistries in serum-free DMEM were added to confluent monolayers of LLCMK-2 cells in 96-well plates for 1 h at 37° C., similar to the focus forming inhibition assays, and subsequently incubated at 37° C. with 5% (v/v) CO₂ for 24 h. Absorbance at 560 μm was measured using a Tecan GeniosPro plate reader (Tecan US, Durham, N.C.).

### Mechanistic Assays with CF 238

Post-Entry Focus-Forming Assay with CF 238 Against DENV-2

To determine if the observed inhibitory effect was due to interference with post-entry steps in the viral life cycle, approximately 200 FFU of DENV-2 without CF 238 was allowed to bind and enter target cells for 1 h at 37° C. as described for the focus forming assay. Unbound virus was then removed by rinsing with PBS and CF 238 was added to the cells post-entry for 1 hr at 37° C. Cultures were washed again in PBS and agarose overlays, incubation, and immunological detection was conducted as described for the focus forming assay.

Pre-Binding Focus-Forming Assay with CF 238 Against DENV-2

To determine if the observed inhibitory effect was due to interference caused by modifications to the target cell surface, CF 238 was incubated with the target cells for 1 h at 4° C., the cells were rinsed with PBS, and approximately 200 FFU of DENV-2 was allowed to infect the cells at 4° C. Agarose overlays, incubation, and immunological detection were conducted as described for the focus forming assay.

Post-Binding Focus-Forming Assay with CF 238 Against DENV-2

To determine if the observed inhibitory effect was due to interference with interactions that occur pre-binding versus post-binding of virions to the target cells, approximately 200 FFU of DENV-2 was allowed to bind to target LLCMK-2 cells for 1 h at 4° C. to allow binding, but prevent internalization. Unbound virus was washed off with PBS at 4° C., then CF 238 was added and incubated at 4° C. for 1 h. Cultures were washed again in 4° C. PBS and warmed to 37° C. Agarose overlays, incubation, and immunological detection were conducted as described for the focus forming assay.

qRT-PCR Virus Binding Assay

Infection of LLCMK-2 target cells in six well plates was performed in duplicate using 10⁵ FFU of DENV-2 that had been pre-incubated for 45 m at 4° C. with CF 238 or pooled heterotypic anti-DENY human serum. After a 45 m infection at 4° C., infected monolayers were washed with PBS and harvested with a cell scraper, added to a 1.5 ml microfuge tube containing 350 μl of AR-200 silicone oil (Sigma-Aldrich, St. Louis, Mo.) mixed with 150 μl of silicone fluid (Thomas, Swedesboro, N.J.), and spun at 14,000 rpm in a microfuge for 1 m to separate the unbound virus from the cell-bound virus in the pellets. The tubes were then submerged in liquid nitrogen for 30 s to freeze the contents. The cell pellets with bound virus were recovered by clipping off the bottoms of the tubes with small wire clippers into 15 ml conical tubes. Viral RNA was extracted from the cell pellets using the Qiagen Viral RNA Extraction kit (Qiagen, Chatsworth, Calif.).

Quantitative real time reverse transcription PCR (qRT-PCR) was performed on the extracted RNA using the Quantitect Sybr Green RT-PCR kit (Qiagen inc., Chatsworth, Calif.), following the manufacturer's specifications and amplification protocols, using dengue-specific primers: (Den2F: catatgggtggaatctagtacg, Den2R: catatgggtggaatctagtacg). Each reaction was performed in 20 μL total volume (10 μL 2×SYBR green master mix, 0.5 μL of 10 μM of each primer, 0.2 μL reverse transcriptase, and 5 μL viral RNA) using a Lightcycler thermal cycler (Roche Diagnostics, Carlsbad, Calif.), and according to the following amplification protocol: 50° C. for 20 min to reverse transcribe the RNA; 95° C. for 15 min to activate the HotStart Taq DNA Polymerase; 45 PCR cycles: 94° C. for 15 s, 50° C. for 15 s, 72° C. for 30 s, the last step was also the fluorescence data acquisition step. Melting curve analysis was performed by a slow increase in temperature (0.1° C./s) up to 95° C. The threshold cycle, representing the number of cycles at which the fluorescence of the amplified product was significantly above background, was calculated using Lightcycler 5.3.2 software (Roche).

### Analysis

Figures were generated using the Origin 6.0 graphing software (Northampton, Mass.). Statistical analyses were performed using the Graphpad Prism 4.0 software package (San Diego, Calif.). P values less than 0.05 were considered significant.

### Results

Inhibition Assays with Different Chemistries Against DENV-2

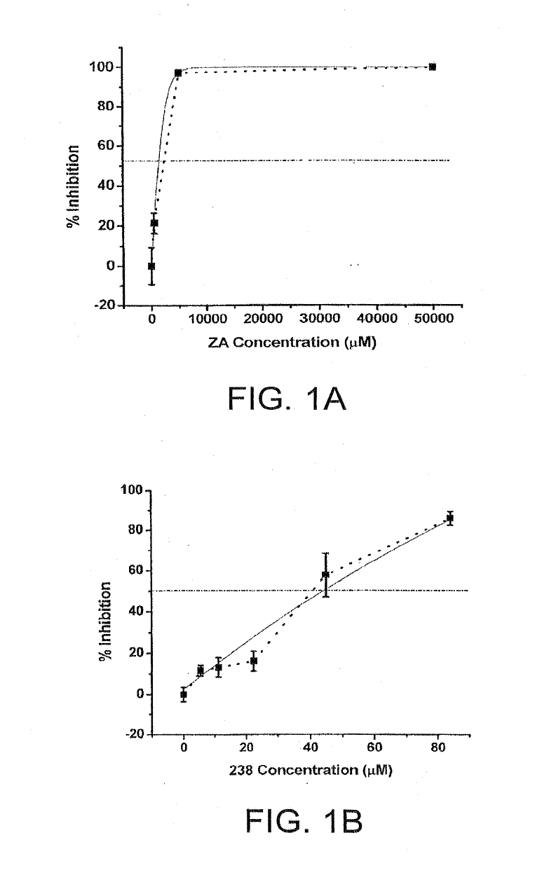

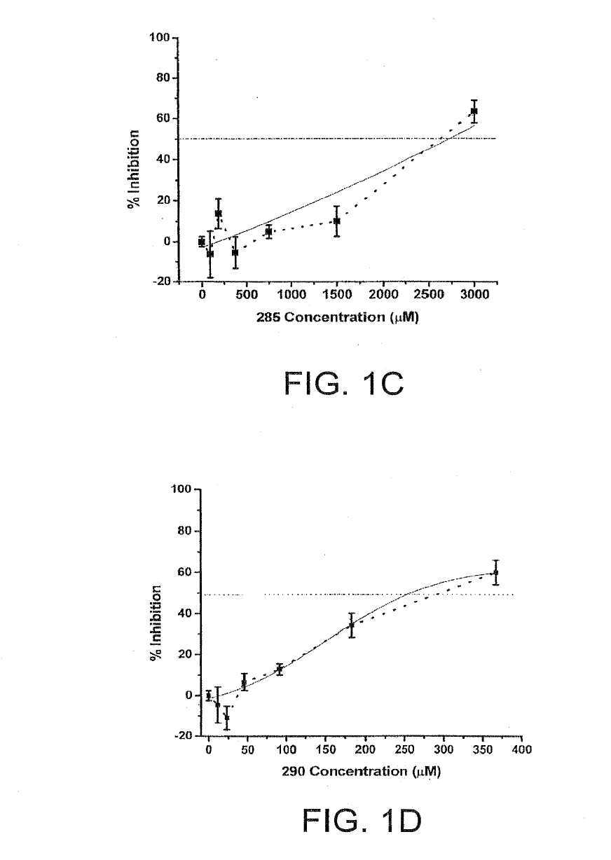

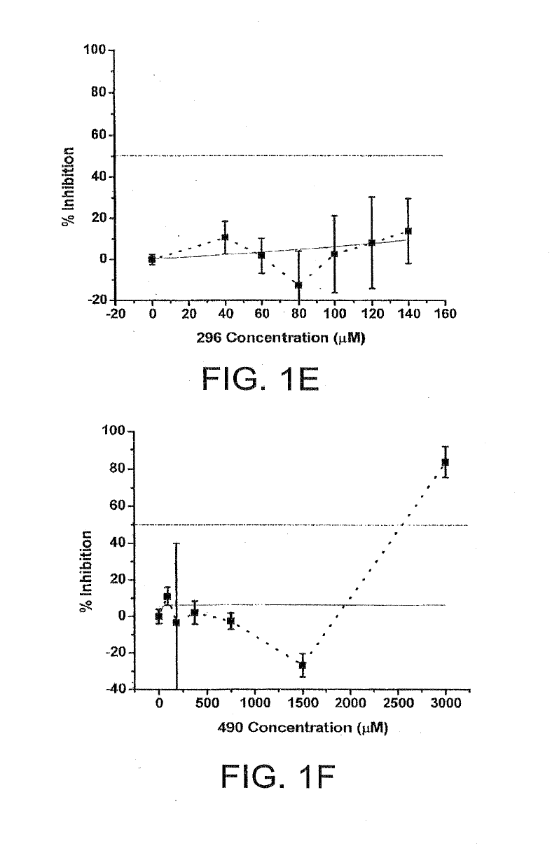

Focus-forming assays were used to quantitate the inhibitory activities of each chemistry against DENV-2. As seen in FIGS. 1A-1-F, dose response curves were generated over concentration ranges dictated by the solubilities of the chemistries in 1% DMSO/aqueous solution. Control 1% DMSO/PBS solutions showed no DENV inhibitory activity in this assay system (data not shown). The natural product, ZA showed a dose response inhibition with an IC₅₀ of 2,380±150 μM±sem, and at 5,000 μM ZA showed 99% inhibition. Two other chemistries, CF 285 and CF 490, showed inhibition curves with IC₅₀s similar to ZA (2,516±172 and 2,378±192 μM, respectively). CF 285 showed a maximum inhibition of 63% at 3,000 μM and CF 490 showed a maximum inhibition of 84% at 3,000 μM. Another compound, CF 290, showed an inhibition curve with an IC₅₀ of 294±42. CF 290 showed a maximum inhibition of 60% at 367 μM. The most active chemistry, CF 238, showed an IC₅₀ of 46±4 μM and achieved 86% inhibition at 84 μM. CF 296 did not show evidence of clear dose-dependent inhibition against DENV-2.

Inhibition Assays with CF 238 Against DENV-1, 3, and 4

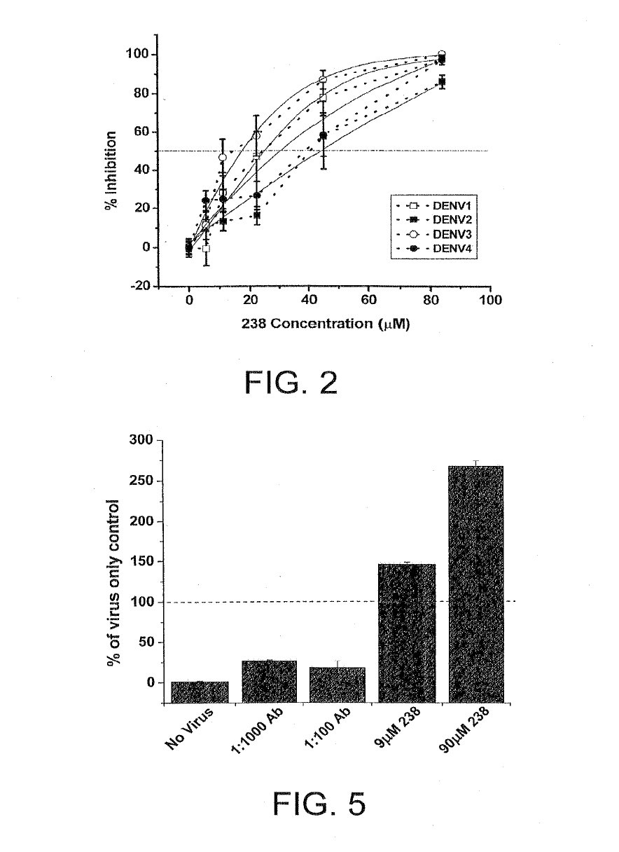

Dose-response inhibition curves were generated for the most active chemistry, CF 238, against the other three strains of dengue virus, resulting in similar overall inhibitory effects against all four strains of dengue virus are shown in FIG. 2. CF 238 showed IC₅₀ values of 24±6, 14±2 and 47±5 μM against DENV-1, DENV-3, and DENV-4, respectively. CF 238 showed consistently high-level inhibition, between 86 and 100%, of all dengue strains at 84 μM.

### Cytotoxicity

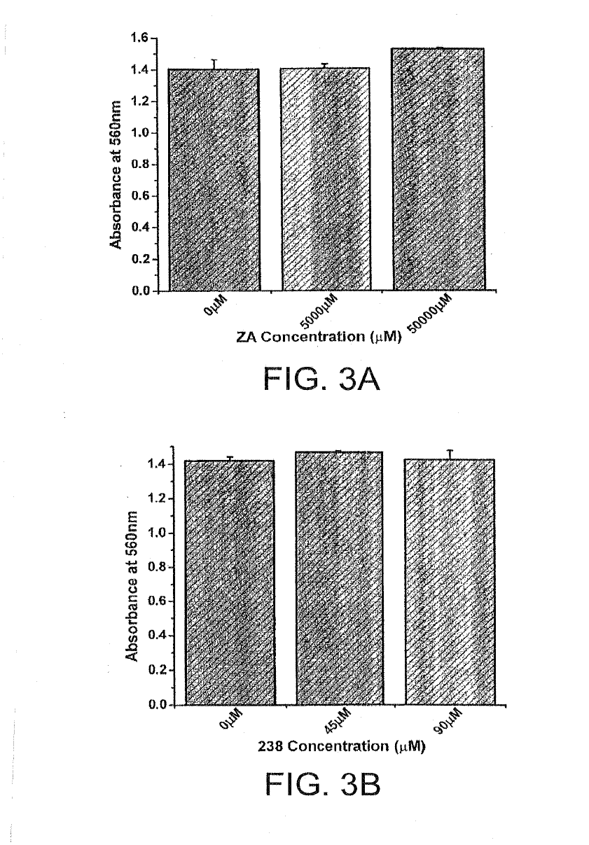

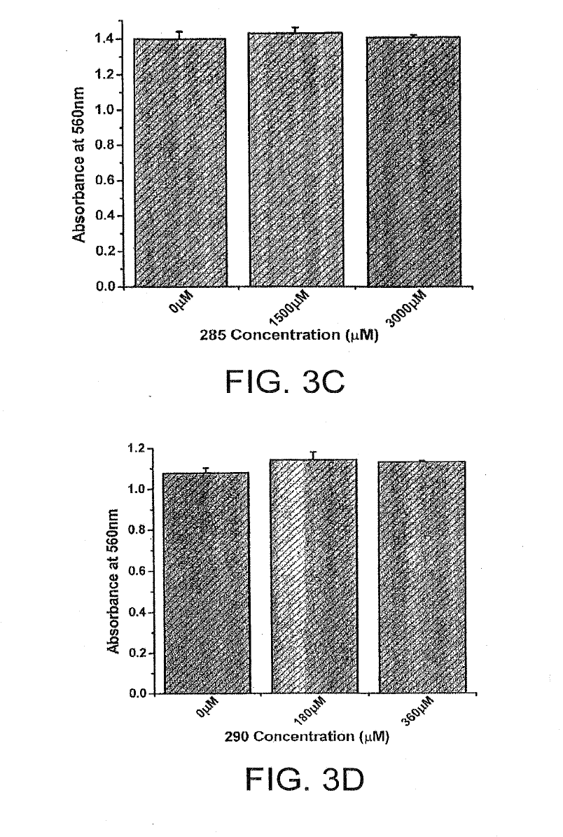

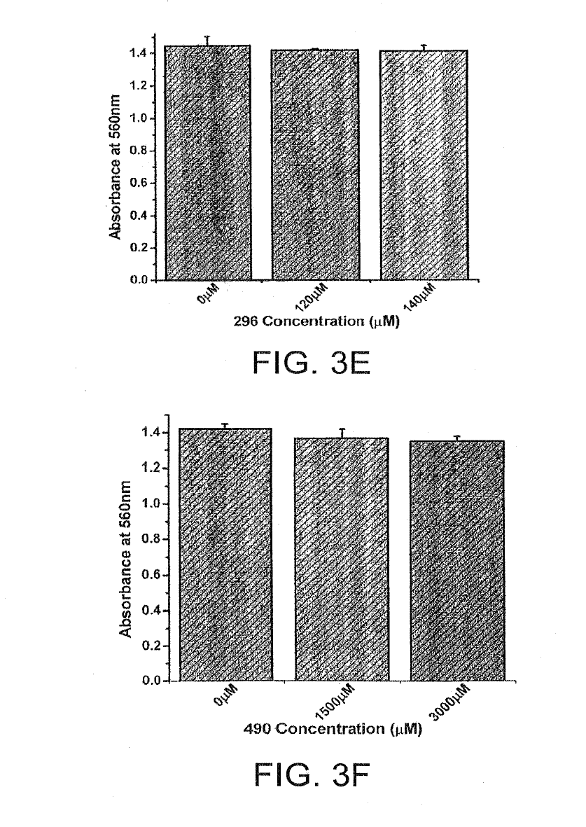

To determine if the observed DENV inhibition effects were due to cellular toxicity that impacted viral replication, the effect of each chemistry on the mitochondrial reductase activity of the target cells over the concentration ranges that showed viral inhibition was measure. In confluent cell monolayers that replicated the conditions in the focus forming assays, there were no observed signs of toxicity with any compound compared to medium only controls (p>0.05, ANOVA with Dunnett's posthoc test) as seen in FIGS. 3A-3F.

### Mechanistic Assays

To investigate the mechanism of action of the inhibitory activity of the most active compound, a series of assays designed to identify the stage at which CF 238 exerts its effects against DENV-2 were conducted.

Post-Entry Focus-Forming Assay with CF 238 Against DENV-2

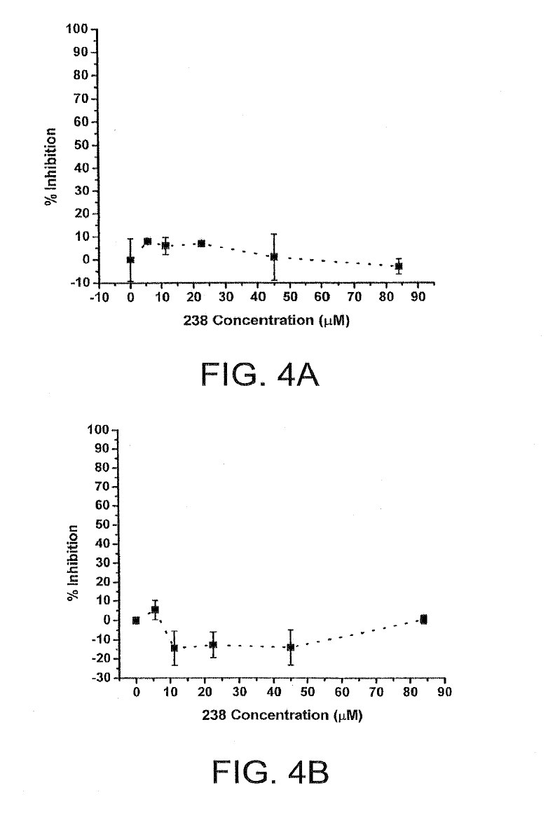

In this assay, CF 238 was added to target cells that had already been infected for 1 h with DENV-2 in order to determine if CF 238 functions during an entry or a post-entry step in the virus life cycle. As seen in FIG. 4A, treatment of DENV-2 infected cells with CF 238 after viral entry resulted in no evidence of inhibition. This indicates that CF 238 inhibits an entry step as opposed to a post-entry step.

Pre-Binding Focus-Forming Assay with CF 238 Against DENV-2

In this assay, CF 238 was added to target cells for 1 h prior to infection with DENV-2 to determine if CF 238 inhibits entry through interaction directly with the target cells. Treatment of target cells with CF 238 prior to DENV-2 infection resulted in no evidence of inhibition as shown in FIG. 4B, indicating that CF 238 does not function by interacting with or modifying the target cell surface, and must be present along with the virus in order to inhibit entry.

Post-Binding Focus-Forming Assay with CF 238 Against DENV-2

In this assay, DENV-2 was added to target cells at 4° C. to bind virus to the surface of target cells, but prevent internalization. CF 238 was then added to determine if CF 238 could dislodge bound virus from the cells. No inhibition of infection was observed under these conditions, over the concentration range that showed inhibition when virus and CF 238 were pre-incubated and added together as seen in FIG. 4. This indicates that CF 238 is not capable of inhibiting virus that is already bound to a target cell surface. This suggests that CF 238 interferes with an early step in entry, prior to permissive binding, endocytosis, or fusion.

qRT-PCR Virus Binding Assay with CF 238 Against DENV-2

In order to directly test if CF 238 interferes with virus binding to target cells, binding assays using qRT-PCR were conducted to monitor attachment of virus to target cells. In these experiments, virus was co-incubated with CF 238 for 45 m at 4° C. and used to infect target cells at 4° C. for 45 m. The cells were then scraped off the plates and centrifuged through an oil mixture with a density that allowed passage of the cells, but not free virus, to the bottom of the tube. RNA was then extracted from the cell pellets and amplified with DENV-2 specific primers. Pre-incubation of DENV-2 with CF 238 did not inhibit virus binding, as measured by the qRT-PCR signal, whereas pre-incubation of DENV-2 with pooled human heterotypic anti-DENV-2 serum resulted in a large decrease in the attachment of virus to target cells, as seen in FIG. 5. This indicates that CF 238 does not prevent virus binding/attachment to target cells under the experimental condition tested.

The results from the tests, as described herein, reveal anti-viral activities of zosteric acid and two of the combinatorial compounds, CF 285 and CF 490, having IC₅₀s of approximately 2 mM. It is believed that the sulfoxy group of the zosteric acid may play a role in the DENY inhibition as seen with other compounds including heparan sulfate and other sulfated polysaccharides as DENV entry factors and entry inhibitors. However, two other compounds without sulfoxy groups, CF 290 and CF 238, were found to be substantially more active from an inhibition standpoint, with IC₅₀s of 294 and 46 μM against DENV-2, respectively. The highest concentrations tested were constrained by the aqueous solubility of the compounds and none of the compounds were toxic to cultures of target epithelial cells over the range of concentrations where viral inhibition was observed. CF 238 also showed similar activity against the other three DENV types with IC₅₀s between 14 and 47 μM.

As determined through analysis of the data, it appears that post-infection treatment of cells with CF 238 inhibits DENV at a viral entry step, as opposed to a later step in replication. It also appears that CF 238 does not inhibit virus infection when pre-incubated with target cells, indicating an activity dependent upon interactions with the virions. qRT-PCR analysis of virus:cell binding reveals that CF 238 does not substantially interfere with virus binding to target cells, but instead enhances virus:cell binding. It is envisioned that CF 238 may not inhibit or dislodge the virus that has been previously bound to target cells at 4° C. and may not inhibit virus E protein mediated agglutination of red blood cells. This result is unexpected since conventional thought that preventing virus:cell binding would cause inhibition and that promoting virus:cell binding would cause increased infection. As the data shows, this is not the case since inhibition of infection associated with enhanced virus:cell binding is observed.

Since CF 238 does not interfere with virus binding to either permissive epithelial host cells (LLCMK-2s) or red blood cells, it is believed that CF 238 may function by tethering or trapping the virus in some inappropriate conformation on the target cell surface. Therefore, it is envisioned that in order to initiate a productive infection, viruses must bind to target cells in a permissive manner and that, alternatively, non-permissive binding modes may exist. With this rationale, CF 238 may therefore function by tethering or trapping the virus in a manner on the target cell surface. Similarly, these chemistries might then be useful in surface tethered configurations for trapping other pathogens. This may include the virus attached to cells in such a way that it is prohibited from gaining access to primary and/or secondary receptor molecules that are required for productive entry. Thus, these chemistries may be useful reagents for probing the interactions between DENV and entry receptor molecules. Similarly, some dimeric, as well as multimeric chemistries, such as those discussed herein, may have a single surface adherent or multi-surface tethering activities. These chemistries might then be useful in certain tethered configurations for trapping pathogens to make them non-infectious or for detection purposes. In this regard, CF 238 may also be a useful reagent for the study of DENV entry mechanisms since it may prevent interactions with virus receptors.

Furthermore, it is also possible that CF 238 may inhibit entry by interfering with some step in the fusion process as is the case for some DENV inhibitory peptides. A potential mechanism of action where CF 238 interferes with entry of the virus by substantially preventing virus:cell contacts may occur when these compounds function through binding to attachment domains on adherent organisms and subsequent release from the protected surface.

It may also be possible to assign functional significance to the chemical structures of the combinatorial chemistries with greater or lesser anti-viral activity. Since CF 238 is on the order of 100 times more active than the original natural product, zosteric acid, additional combinatorial chemistries may identify molecules with even greater inhibitory activities against DENY or other viruses.

Various applications utilizing zosteric acid and the other related molecules, as shown in Table 1, may be employed in preventing a viral outbreak as well as protecting individuals from contracting and dispersing a viral contaminant.

In one embodiment of the invention, at least one anti-viral compound, such as shown in Table 1, may be coated onto the surface of a substrate. A suitable substrate may include a metal substrate. In one embodiment of the invention, the metal substrate may include a metal sheet, metal foil, metal wool and a powdered metal. The coated metal substrate may include the at least one compound covalently linked a surface of the metal substrate. Covalently linking the compound to the metal substrate may provide a way to tether, trap or capture a virus once it comes in contact with the coated metal substrate. Applications for the coated metal substrate include insertion within air handling and treatment systems such as heating, ventilation and air conditioning systems. Any other suitable environment or structure could also be coated or otherwise provided with the anti-viral compound(s) to provide treatment of fluids, including gases or liquids.

In another embodiment of the invention, at least one compound as shown in Table 1 may be applied to a surface in a form that requires activation in order to provide anti-viral inhibition. For example, a solution that includes at least one compound as shown in Table 1 may be applied, for example by spraying, onto a surface, such as the walls and floor of a building or a container. Once the solution has dried, that is the solvent has evaporated from the solution, the at least one compound may remain on the surface in an inactive state. The at least one compound may be activated when an activating agent, such as a polar material including water, solubilizes the at least compound making it available to tether, trap or capture a virus once it comes in contact with the activated compound.

In another embodiment of the invention, a solution containing at least one compound as shown in Table 1 may be encapsulated in a degradable housing and applied to a porous substrate. Suitable porous substrates may include concrete, adobe or mud walls and dry wall. The encapsulated solution containing the at least one compound as shown in Table 1 may become available after the porous substrate is contacted either through gradual wearing or immediate contact of the degradable housing. The at least one compound contained within the solution within the porous substrate may then be able to tether, trap, adhere to or capture a virus if present.

In yet another embodiment of the invention, a disposable respiratory mask or a filter medium may be provided with the materials according to the invention integrated therein for treatment of fluids or gases. For example, a disposable respiratory mask may be provided to be worn by individuals who may be working in or susceptible to contacting a virus to be protected against, or a person infected with a virus could wear such as mask to prevent transmission of the virus to others. In this example, the mask may be coated or impregnated with a solution that contains at least one anti-viral compound, such as shown in Table 1. In one embodiment of the invention, the disposable respiratory mask is porous to allow transmission of air therethrough and provide the ability for the user to breathe in a normal fashion. In order to ensure viable protection from a virus over an extended time period, the respiratory mask and the filter may be sprayed or otherwise coated, initially before wearing and/or at one or more times during wear, with a solution containing at least one compound as shown in Table 1. In use, if a virus is encountered by a user, and is attempted to be breathed in or is exhaled by the user, the compound on the mask will be encountered and the virus will be effectively adhered to the compound, such that transmission to or from the user is prevented. Similarly, in a filter medium, any acceptable filter medium may be coated, or impregnated or otherwise suitably provided with at least one anti-viral compound, such as shown in Table 1. The filter media may then be positioned in a suitable location to effectively filter fluids passing therethrough, such as air or liquid materials. Similar to the respiratory mask, the filter materials or medium can be porous to allow transmission of gases and/or liquids therethrough, and provide the ability for any virus contained in the fluid to contact the anti-viral compound(s) in the filter to be tethered, trapped, adhered to or captured as the fluid moves through the filter.

In another embodiment, the surfaces or structures, the respiratory mask and/or the filter type products may be coated with a gelatinous composition that contains at least one anti-viral compound, such as shown in Table 1. The gelatinous composition may facilitate creation of the suitable environment for the interaction between the anti-viral compound(s) and the virus encountered, providing a long lasting effect when applied to a medium such as a respiratory mask or filter media for example. In the example of a coated mask, this again may provide dual protection when a viral outbreak occurs. In one embodiment, the coated mask may be worn by medical personnel who are treating individuals exposed to a virus. In another embodiment, the coated mask may be worn by individuals who have contracted a virus and may be used to limit the exposure of other individuals to the virus.

Based upon the foregoing disclosure, it should now be apparent that the use of inhibitors that interact with regions of a virus, such as the dengue virus E protein, may be useful as potential candidates for the development of anti-viral compounds as described herein will carry out the objects set forth hereinabove. It is, therefore, to be understood that any variations evident fall within the scope of the claimed invention and thus, the selection of specific component elements can be determined without departing from the spirit of the invention herein disclosed and described.

What is claimed is:

1. An anti-viral compound comprising:
a chemical compound represented by general structure: 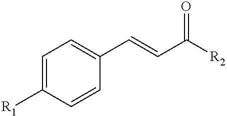
wherein,
R₁ represents —OH or —OSO₂OH;
R₂ represents —OH, optionally substituted alkyl, heteroalkyl, cycloalkyl, heterocycloalkyl, aryl, heteroaryl, aralkyl, or heteroaralkyl.

2. The compound of claim 1, wherein the compound is zosteric acid which is represented by general structure:

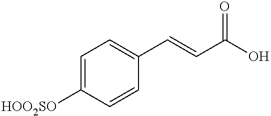

3. The compound of claim 1, wherein the compound is CF 238 which is represented by general structure:

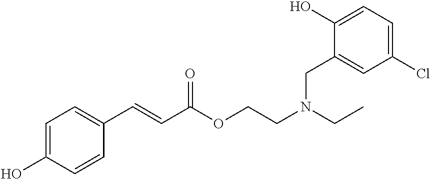

4. The compound of claim 1, wherein the compound interacts with a portion of a class II E protein.

5. The compound of claim 4, wherein the class II E protein is a dengue virus E protein.

6. The compound of claim 4, wherein the interaction of the compound to the class II E protein reduces viral infectivity.

7. The compound of claim 6, wherein the interaction of the compound to the class II E protein inhibits viral infectivity.

8. The compound of claim 1, wherein the compound is linked to a carrier molecule.

9. The compound of claim 8, wherein the carrier molecule is a protein.

10. The compound of claim 9, wherein the protein is human serum albumen.

11. The compound of claim 1, wherein the compound inhibits dengue virus virion:cell fusion.

12. The compound of claim 1, wherein the compound is administered in a pharmaceutical carrier.

13. The compound of claim 12, wherein the pharmaceutical carrier is selected from the group consisting of saline, buffered saline, dextrose and water.

14. The compound of claim 1, wherein the compound is administered to a patient from the group consisting of intradermally, intramuscularly, intraperitoneally, intravenously, subcutaneously, orally and intranasally.

15. A substrate having at least one surface coated with the compound of claim 1.

16. The substrate of claim 15, wherein the substrate is selected from the group consisting of a metal sheet, a metal foil, metal wool and a powdered metal.

17. The substrate of claim 15, wherein the compound is covalently linked to the at least one surface of the substrate.

18. The substrate of claim 15, wherein the substrate is insertable within air handling and treatment systems selected from the group consisting of heating, ventilation and air conditioning systems.

19. The substrate of claim 15, wherein the substrate provides treatment of fluids selected from the group consisting of gases and fluids.

20. The substrate of claim 15, wherein the substrate is selected from the group consisting of walls and floors of a building or a container.

21. The substrate of claim 20, wherein the substrate is sprayed with a solution of the compound.

22. The substrate of claim 21, wherein the solution of the compound evaporates and the compound remains of the surface in an inactive state.

23. The substrate of claim 22, wherein an activating agent activates the compound allowing the compound to tether, trap or capture a virus once the virus contacts the activated compound.

24. A solution containing the compound of claim 1 encapsulated in a degradable housing, wherein the encapsulated solution is applied to a porous substrate.

25. The solution of claim 24, wherein gradual wearing or immediate contact of the degradable housing provides accessibility the solution containing the compound.

26. The solution of claim 25, wherein the solution containing the compound is capable of tethering, trapping or capturing a virus.

27. A filter medium comprising the compound of claim 1.

28. The filter medium of claim 27, wherein the filter medium is a porous respiratory mask.

29. The filter medium of claim 27, wherein fluids or gases are treated with the filter medium.

30. The filter medium of claim 27, wherein the filter medium is coated with a solution containing the compound.

31. The filter medium of claim 30, wherein the compound is capable of tethering, trapping or capturing a virus within the filter medium.

32. The filter medium of claim 27, wherein the filter medium is coated with a gelatinous composition containing the compound.

33. The filter medium of claim 32, wherein the filter medium is a porous respiratory mask.

34. The filter medium of claim 32, wherein the compound is capable of tethering, trapping or capturing a virus within the filter medium.

35. A method for inhibiting viral infection, the method comprising the steps of:
providing a compound that interacts with a region of a virus, wherein the compound is represented by general structure: 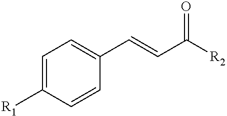
wherein,
R₁ represents —OH or —OSO₂OH;
R₂ represents —OH, optionally substituted alkyl, heteroalkyl, cycloalkyl, heterocycloalkyl, aryl, heteroaryl, aralkyl, or heteroaralkyl.

36. The method of claim 35, wherein the compound is zosteric acid which is represented by general structure:

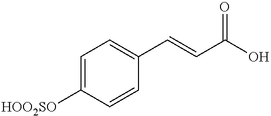

37. The method of claim 35, wherein the compound is CF 238 which is represented by general structure:

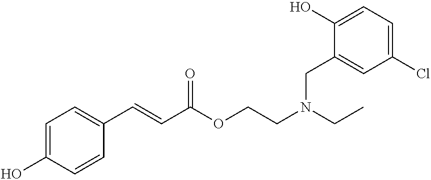

38. The method of claim 35, wherein the virus is a virus selected from the group consisting of a dengue virus, an influenza virus or a human immunodeficiency virus.

39. The method of claim 35, wherein the compound inhibits entry of the virus into a cell, prior to permissive binding, endocytosis, or fusion.

40. The method of claim 40, wherein the compound is capable of tethering or trapping the virus on a cell surface.

41. The method of claim 35, wherein the compound is externally provided onto a substrate or medium.

42. The method of claim 35, wherein the compound is internally provided within a body of a patient.
